(19) United States
(12) Patent Application Publication
(10) Pub. No.: US 2020/0009078 A1
(43) Pub. Date: Jan. 9, 2020

(54) ORAL COMPOSITIONS

(71) Applicant: J. Phillip KENNEDY, Grand Rapids, MI (US)

(72) Inventor: J. Phillip KENNEDY, Grand Rapids, MI (US)

(73) Assignee: TRUETIVA, Inc., Grand Rapids, MI (US)

(21) Appl. No.: 16/502,244

(22) Filed: Jul. 3, 2019

Related U.S. Application Data
(60) Provisional application No. 62/693,703, filed on Jul. 3, 2018.
(60) Provisional application No. 62/702,936, filed on Jul. 25, 2018.
(60) Provisional application No. 62/804,240, filed on Feb. 12, 2019.

(51) Int. Cl. A61K 31/05; A61K 9/00; A61K 31/353; A61K 31/4525

(52) U.S. Cl. CPC A61K 31/05; A61K 31/4525; A61K 31/353; A61K 9/0053

(57) ABSTRACT

Embodiments of the invention are directed to compositions containing cannabinoid, cannabidiol, cannabidiol isomer, or cannabidiol analog and combinations thereof for treating dermatological disease, and methods for treating dermatological diseases by administering compositions containing cannabinoid, cannabidiol, or cannabidiol analog to the skin of a patient in need of treatment.

### CROSS-REFERENCE TO RELATED APPLICATIONS

This application claims priority from U.S. Provisional No. 62/693,703 entitled “Compositions for Treating Dermatological Diseases,” filed on Jul. 3, 2018, U.S. Provisional No. 62/702,936 entitled “Compositions for Treating Dermatological Diseases,” filed Jul. 25, 2018, and U.S. Provisional No. 62/804,240 entitled “Compositions for Enhanced Bioavailability and Methods for Same” filed on Feb. 12, 2019, each of which are hereby incorporated by reference in their entireties.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Not applicable

### SUMMARY OF THE INVENTION

Various embodiments are directed to compositions containing a cannabinoid, a bioenhancer having a concentration of about 0.05% w/w to about 20% w/w, relative to the total weight of the composition, and a pharmaceutically acceptable carrier, excipient, diluent, reagent, or combinations thereof. In some embodiments, the cannabinoid may have a concentration of about 1% (w/w) to about 20% (w/w) to cannabinoid, relative to the total amount of the composition. In some embodiments, the cannabinoid may be cannabidiol, cannabidiol isomer, cannabidiol analog, and the like and combinations thereof. In certain embodiments, the bioenhancer may be a P-glycoprotein inhibitor such as piperine, quercetin, genistein, naringin, sinomenine, glycyrrhizin, nitrile glycosides cuminum cyminum, zingiver officinale, lysergol, allium sativum, aloe vera, liposomes, microspheres, nanoparticles, transferosomes, ethosomes, nanoemulsions, microemulsions, lipid based systems, polymeric micelle formulations, ketoprofen-loaded solid lipid nanoparticles, beeswax, carnauba wax, natural waxes, solid lipids, Ginkgo biloba, lipid-based systems, silybin lipid-based systems, ginseng lipid-based systems, hawthorn lipid-based systems, quercetin lipid-based systems, curcumin lipid-based systems, capsaicin transferosomes, colchicine tranferosomes, vincristine tranferosomes, and the like and combinations thereof.

In some embodiments, the composition may further include a steroid, anti-inflammatory, immune checkpoint blockade inhibitor, antibiotic, antiviral compound, and the like and combinations thereof. In certain embodiments, the amount of steroid, anti-inflammatory, immune checkpoint blockade inhibitor, antibiotic, antiviral compound, and the like or combinations thereof have a concentration of about 0.01% to about 50% (wt/wt), relative to the total amount of the composition.

In various embodiments, the composition may be in the form of a gel, tonic, tincture, pills, tablet, capsule, or combinations thereof.

Other embodiments are directed to methods for treating a disease by administering to a patient in need of treatment composition comprising a cannabinoid having a concentration, a bioenhancer having a concentration of about 0.05% wt to about 20% wt, relative to the total weight of the composition, and a pharmaceutically acceptable carrier, excipient, diluent, reagent, or combinations thereof. In some embodiments, the disease may be, for example, inflammatory disease, autoimmune disease, heart disease, obesity, and combinations thereof. In certain embodiments, the disease may be, for example, arteriosclerotic heart disease, arthritis, rheumatoid arthritis, asthma, gout, attention deficit hyperactivity disorder (ADD/ADHD), autism, Ssperger's syndrome, chronic pain, substance dependence, diabetic neuropathy, glaucoma, migraine headaches, tension headaches, hypertension, inflammatory autoimmune-mediated arthritis, inflammatory bowel disease (IBD), insomnia, depression, obesity, obsessive compulsive disorder, opiate dependence, osteoarthritis, post-traumatic stress disorder (PTSD), restless legs syndrome (RLS), rheumatoid arthritis, seizures, dementia, nausea, sleep disorders, tourette's syndrome, fibromyalgia, and related conditions, epilepsy, digestive diseases, gliomas, and the like and combinations thereof.

In some embodiments, the cannabinoid may have a concentration of about 1% (w/w) to about 20% (w/w) to cannabinoid, relative to the total amount of the composition. In some embodiments, the cannabinoid may be cannabidiol, cannabidiol isomer, cannabidiol analog, and the like and combinations thereof. In certain embodiments, the bioenhancer may be a P-glycoprotein inhibitor such as piperine, quercetin, genistein, naringin, sinomenine, glycyrrhizin, nitrile glycosides cuminum cyminum, zingiver officinale, lysergol, allium sativum, aloe vera, liposomes, microspheres, nanoparticles, transferosomes, ethosomes, nanoemulsions, microemulsions, lipid based systems, polymeric micelle formulations, ketoprofen-loaded solid lipid nanoparticles, beeswax, carnauba wax, natural waxes, solid lipids, Ginkgo biloba, lipid-based systems, silybin lipid-based systems, ginseng lipid-based systems, hawthorn lipid-based systems, quercetin lipid-based systems, curcumin lipid-based systems, capsaicin transferosomes, colchicine tranferosomes, vincristine tranferosomes, and the like and combinations thereof.

In some embodiments, the composition may further include a steroid, anti-inflammatory, immune checkpoint blockade inhibitor, antibiotic, antiviral compound, and the like and combinations thereof. In certain embodiments, the amount of steroid, anti-inflammatory, immune checkpoint blockade inhibitor, antibiotic, antiviral compound, and the like or combinations thereof have a concentration of about 0.01% to about 50% (wt/wt), relative to the total amount of the composition.

In various embodiments, the composition may be in the form of a gel, tonic, tincture, pills, tablet, capsule, or combinations thereof.

### DESCRIPTION OF THE DRAWINGS

Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention. In the following description, numerous specific details are set forth in order to provide a thorough understanding of the present invention. The present invention may be practiced without some or all of these specific details. In other instances, well known process operations have not been described in details so as to not unnecessarily obscure the present invention.

FIG. 1 is a comparison of the structure of a PD-L1 dimer bound by BMS-202 (left panel) and cannabidiol (right panel).

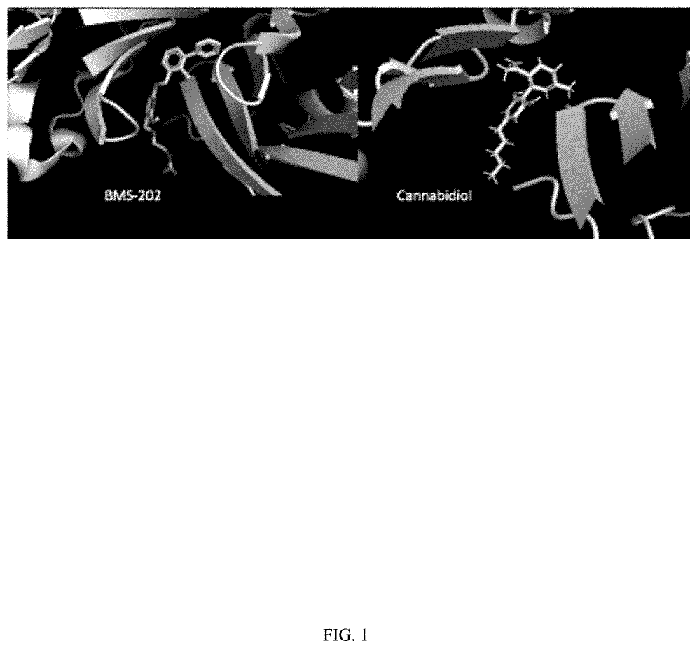

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “disorder” is used in this disclosure to mean, and is used interchangeably with the terms disease, condition, syndrome, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g, animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed compositions and methods for treating a dermatological disease or disorder. Such compositions may include a cannabinoid and the methods may include administering a composition containing cannabinoids to a patient in need of treatment. In particular embodiments, the dermatological disease or disorder may be dermatitis.

The cannabinoids of such embodiments include any of a broad class of compounds that are known to interact with cannabinoid receptors, and encompass endocannabinoids (produced naturally in the body by animals), the phytocannabinoids (found in Cannabis and some other plants), and synthetic cannabinoids (manufactured artificially). Example cannabinoids include, but are not limited to, tetrahydropyran analogs, such as, Δ⁹-tetrahydrocannabinol, Δ⁸-tetrahydrocannabinol, 6,6,9-trimythel-3-pentyl-6H-dibenzo[b,d]pyran-1-ol, 3-(1,1-dimethylheptyl)-6,6a7,8,10,10a-hexahydro-1-1hydroxy-6,6-dimythel-9H-dibezo[b,d]pyran-9-ol, (−)-(3 S,4 S)-7-hydroxy-delta-6-tetrahydrocannabinol-1,1-dim ethylheptyl, (+)-(3 S,4S)-7-hydroxy-A-6-tetrahydrocannabinol, and Δ⁸-tetrahydrocannabinol-11-oic acid, piperidine analogs, such as, (−)-(6S,6aR,9R,10aR)-5,6,6a,7,8,9,10,10a-octahydro-6-methyl-1-3-[(R)-1-methyl-4-phenylbutoxy]-1,9 phenanthridinediol 1-acetate), aminoalkylindole analogs, such as, (R)-(+)-[2,3-dihydro-5-methyl-3-(4-morpholinylm-ethyl)-pyrrolo[1,2,3,-de]-1,4-benzoxazin-6-yl]-1-naphthelenyl-methanone, open pyran-ring analogs, such as, 2-[3-methyl-6-(1-methylethenyl-2-cyclohexen-1-yl]-5-pentyl-1,3-benzendi-ol, and 4-(1,1-dimethylheptyl)-2,3′-dihydroxy-6′-α-(3-hydroxypropyl)-1′,-2′,3 ‘,4’,5′,6′-hexahydrobiphenyl, lipophilic alkylamides, such as, dodeca-2E,4E,8Z,10E/Z-tetraenoic-acid-isobutylamide, cannabinoid mimetics, salts, solvates, metabolites, and metabolic precursors of these compounds and combinations thereof. In some embodiments, the cannabinoids may be derived plants including hemp, Echinacea purpurea, Echinacea angustifolia, Acmella oleracea, Helichrysum umbraculigerum, Radula marginata, and combinations thereof and oils made from these plants, and in other embodiments, the cannabinoids may be manufactured or chemically synthesized.

The compositions of various embodiments can include any number of cannabinoids in various concentrations; however, in certain embodiments, the cannabinoid may be cannabidiol (2-(6-isopropenyl-3-methyl-5-cyclohexen-1-yl)-5-pentyl-1,3-benzenediol). Cannabidiol has 7 double bonds and 30 stereoisomers. Embodiments include compositions containing each stereoisomer individually and compositions containing a combination of these stereoisomers. In particular embodiments, the compositions used in the methods of embodiments and the compositions of embodiments may include high concentrations of cannabidiol. For example, in some embodiments, cannabidiol may be about 30 w/v % to about 100 w/v % of the cannabinoids in the composition, and in other embodiments cannabidiol may be about 50 w/v % to about 100 w/v %, about 75 w/v % to about 100 w/v %, about 80 w/v % to about 100 w/v %, about 90 w/v % to about 100 w/v % of the cannabinoids in the composition.

Cannabidiol can be obtained by cold-pressing industrial hemp with trace amounts of THC. Cannabidiol in this present invention is provided as a natural constituent of hemp oil.

In some embodiments, the cannabinoids in the composition may be cannabidiol analogs. The term “cannabidiol analogs” refers to synthetically produced compounds that are structurally similar, but not structurally identical, to cannabidiol. Various cannabidiol analogs are known in the art and embodiments encompass such cannabidiol analogs. For example, PCT Publication WO2017/132526 and U.S. Pat. No. 6,630,507, which are each hereby incorporated by reference in their entireties, describes various analogs of cannabidiol. In some embodiments, the analogs of cannabidiol may be of general Formula I:

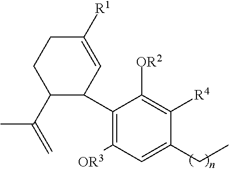

where R¹ is hydrogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are each, individually, hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, R⁴ is hydrogen, substituted or unsubstituted alkyl, carboxyl, alkoxy, aryl, aryloxy, arylalkyl, halo or amino, and n may an integer of 2 to 10 and the like and salts and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof. Like cannabidiol, cannabidiol analogs can have various isomers. Embodiments include all isomers of the such cannabidiol analogs.

In some embodiments, cannabidiol analogs, such as those described above may be combined with cannabidiol, to produce a mixture of cannabidiol and cannabidiol analogs. Thus, as used herein the term “cannabidiol” encompasses cannabidiol, cannabidiol analogs, and the various isomers of cannabidiol and cannabidiol analogs.

The compositions of various embodiments can include up to about 50% (w/w) cannabidiol, cannabidiol analogs, isomers of cannabidiols, and combinations thereof (collectively, “cannabidiol”), relative to the total amount of the composition, and in some embodiments, the compositions may include about 50% (w/w) to about 0.5% (w/w) cannabidiol, relative to the total amount of the composition, about 30% (w/w) to about 1% (w/w) cannabidiol, relative to the total amount of the composition, about 20% (w/w) to about 1% (w/w) cannabidiol, relative to the total amount of the composition, about 20% (w/w) to about 5% (w/w) cannabidiol, relative to the total amount of the composition, or any range or individual concentration encompassed by these example ranges. In particular embodiments, the composition may include about 15% (w/w) to about 10% (w/w) cannabidiol, relative to the total amount of the composition, or any range or individual concentration encompassed by this example range.

In certain embodiments, the cannabidiol of embodiments described above may be cannabidolic acid (“CBDA”). Without wishing to be bound by theory, CBDA may exhibit improved hydrophilicity over other isomers of cannabidiol, which may allow for improved solubility and delivery of CBDA to the skin. The CBDA may be modified, partially digested, or otherwise acted upon by enzymes in the skin to produce for example cannabidiol (CBD), which may be the active form cannabidiol in the composition. Thus, CBDA may act as a prodrug in some embodiments of the invention. Other cannabidiol analogs or isomers may produce a similar effect and are encompassed by prodrug embodiments of the invention.

The cannabidiol in the compositions of embodiments of the invention may be 100% cannabidiol, or oils, solvents, and emulsions containing cannabidiol. For example, in some embodiments, the compositions of the invention may include cannabidiol derived from hempseed oil. Hempseed oil is generally manufactured from varieties of Cannabis sativa that do not contain significant amounts of tetrahydrocannabinol (THC), the psychoactive element of the Cannabis plant. This manufacturing process typically includes cleaning the seed to 99.99% before pressing the oil. Hempseed oil generally also contains omega-6 and omega-3 fatty acids. For example, about 30-35% of the weight of hempseed oil are essential fatty acids (EFAs), i.e., linoleic acid, omega-6 (LA, 55%), α-linolenic acid, omega-3 (ALA, 22%), γ-linolenic acid, omega-6 (GLA, 1-4%), and stearidonic acid, omega-3 (SDA, 0-2%). Thus, the compositions of some embodiments may contain fatty acids such as omega-6 and omega-3 fatty acids.

Oils include cannabidiol oil and various plant derived oils containing cannabidiol, such as, hempseed oil, Echinacea purpurea, Echinacea angustifolia, Acmella oleracea, Helichrysum umbraculigerum, Radula marginata, and the like. In some embodiments, cannabidiol isolated from such plants or made synthetically may be formulated with an oil such as, for example, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and the like and combinations thereof.

Unless indicated otherwise, the term “therapeutically effective amount” is not particularly limited, so long as the cannabinoid is present in an amount effective for treating the dermatological disease. The therapeutically effective amount of cannabinoid can be from about 2 milligrams per kilogram (mg/kg) to about 100 mg/kg, about 2 mg/kg to about 50 mg/kg, about 2 mg/kg to about 25 mg/kg, or any range or individual concentration encompassed by these example ranges, wherein mg refers to the mass or weight of the cannabinoids and kg refers to the mass or weight of the patient in need of treatment. In certain embodiments, a therapeutically effective amount of THC and/or CBD in the composition may be about 2 mg/kg to about 10 mg/kg or any range or individual concentration encompassed by these example ranges, wherein mg refers to the mass or weight of the cannabinoids and kg refers to the mass or weight of the patient in need of treatment.

Without wishing to be bound by theory, the compositions of various embodiments may block inhibitory checkpoints, reducing inflammation in affected areas upon application. Various proteins have been associated with the immune checkpoint blockade including adenosine A2A receptor (A2AR), B7-H3 or CD276, MGA271, B7-H4 or VTCN1, B and T Lymphocyte Attenuator (BTLA or CD272), Cytotoxic T-Lymphocyte-Associated protein 4 (CTLA-4 or CD152), Indoleamine 2,3-dioxygenase (IDO), tryptophan 2,3-dioxygenase (TDO), Killer-cell Immunoglobulin-like Receptor (KIR), Lymphocyte Activation Gene-3 (LAG3), Programmed Death 1 (PD-1), T-cell Immunoglobulin domain and Mucin domain 3 (TIM-3), V-domain Ig suppressor of T cell activation (VISTA). The cannabidiols of embodiments may block activity of these or other immune checkpoint blockade proteins, reducing immune response and improving the symptoms of dermatitis. For example, the cannabidiols of the compositions of the invention may impact the interaction between transmembrane protein PD-1 and its ligands, PD-1 ligand 1 (PD-L1) and PD-1 ligand 2 (PD-L2) by blocking binding of PD-L1 or PD-L2 with PD-1. Inhibiting the activity of PD-1 may reduce T-cell signaling, preventing the immune response and reducing inflammation in the affected area and reducing the symptoms of dermatitis.

Inhibitory checkpoints like PD-1 and PD-L1 have been linked to cancer. For example, cancer-mediated upregulation of PD-L1 on the cell surface may inhibit T cells that might otherwise attack a tumor or other tumorigenic tissue. Thus, the compositions of various embodiments can be used as anti-cancer agents to block the interaction PD-1 or PD-L1, allowing T-cells to attack the tumors or tumorigenic tissue. In some embodiments, the cancers may be one or more of melanoma, lung cancer, pancreatic cancer, kidney cancer and Hodgkin's lymphoma, for example.

In various embodiments, the cannabidiol containing compositions described above may include one or more additional immune checkpoint blockade inhibitors or the cannabidiol containing compounds of the invention may be administered in combination with one or more additional immune checkpoint blockade inhibitors. Additional checkpoint blockade inhibitors include, for example, an IgG4 PD1 antibody such as, for example, antibody BGB-A317, Nivolumab, or Pembrolizumab, a PD-L1 inhibitor, such as, for example, atezolizumab, avelumab, or and durvalumab, antibodies that block the immune checkpoint molecule CTLA-4 such as, for example, ipilimumab, therapeutic agents that target an intrinsic checkpoint blockade, such as, for example, the gene encoding Cytokine-inducible SH₂-containing protein (CISH). The amount of immune checkpoint blockade inhibitor in the compositions may be from about 0.01% to about 5% (w/w), relative to the total amount of the composition, from about 0.1% to about 1% (w/w), relative to the total amount of the composition, or any range or individual concentration encompassed by these example ranges.

The cannabinoids discussed above can be combined with various additional agents to improve efficacy of treatment, aid in delivery of the cannabinoid, or treat additional symptoms associated with the dermatological disease.

For example, in some embodiments, the compositions may include one or more bioenhancers. Bioenhancers include any compound or composition that aids in the transport of another compound across epithelial membranes. Bioenhancers include P-glycoprotein inhibitors, compounds that reverse P-glycoprotein-mediated efflux, limit metabolism of active agents, increase gastric emptying time and intestinal motility, reduce degradation of the active agent by hydrochloric acid, modify cell membrane permeability, produce a cholagogue effect, modify the bioenergetics and thermogenic properties of the active agent, suppress first pass metabolism, and inhibit metabolizing enzymes, stimulate gamma glutamyl transpeptidase, enhance the uptake of amino acids, and the like and combinations thereof. In some embodiments, the bioenhancers may be herbal or neutraceutical bioenhancers. Examples of bioenhancers encompassed by the invention include piperine, quercetin, genistein, naringin, sinomenine, glycyrrhizin, nitrile glycosides cuminum cyminum, zingiver officinale, lysergol, allium sativum, aloe vera, and the like and combinations thereof. In some embodiments, the bioenhancers may be liposomes, microspheres, nanoparticles, transferosomes, ethosomes, nanoemulsions, microemulsions, lipid based systems, polymeric micelle formulations, ketoprofen-loaded solid lipid nanoparticles, and the like, which can be made from beeswax, carnauba wax, or other natural waxes and solid lipids, and combinations thereof. In some embodiments, the bioenhancers may be liposomal enhancers such as, for example, Ginkgo biloba lipid-based systems, silybin lipid-based systems, ginseng lipid-based systems, hawthorn lipid-based systems, quercetin lipid-based systems, curcumin lipid-based systems, and the like and combinations thereof. In further embodiments, the bioenhancers may be capsaicin transferosomes, colchicine tranferosomes, vincristine tranferosomes, and the like and combinations thereof, which may find particular use as natural skin penetration agents.

The amount of bioenhancer in the compositions may be from about 0.05% to about 20% (w/w), relative to the total amount of the composition or in some embodiments, from about 0.1% to about 10% (w/w), relative to the total weight of the composition, from about 0.1% to about 5% (w/w), relative to the total amount of the composition, from about 0.1% to about 2% (w/w), relative to the total amount of the composition, or any range or individual concentration encompassed by these example ranges.

For example, piperine enhances bioavailability by modulating DNA receptor binding and cell signal transduction, while inhibiting efflux pumps that remove the active agent from cells. This inhibits drug metabolizing enzymes and stimulates absorption by stimulating gut amino acid transporters and inhibiting cellular pumps responsible for drug elimination from cells and intestinal production of glucuronic acid. Piperine also increases the absorption of the active agent in the gastrointestinal tract and inhibits enzymes responsible drug metabolism especially in the liver during first pass metabolism such as hepatic arylhydrocarbon hydrolase and UDP-glucuronyltransferase activities. Piperine modifies the rate of glucuronidation by lowering the endogenous UDP-glucuronic acid content and also by inhibiting transferase activity. Piperine inhibits P-glycoprotein and cutochrome P450 3A4, also CYP1A1, CYP1B1, CYP1B2, CYP2E1, CYP3A4, among others and makes target receptors more responsive to drugs, acting as receptors for drug molecules, increasing GIT vasculature by vasodilation to increase the absorption of drugs, modulation of cell membrane dynamics which increases transport of drugs across the cell membranes.

In some embodiments, the compositions may further include hydrocortisone or any steroid within Groups I to VII in the US classification system. Group I steroids include, but are not limited to, clobetasol propionate, betamethasone dipropionate, halobetasol, and diflorasone diacetate. Group II steroids include, but are not limited to, fluocinonide, halcinonide, amcinonide, and desoximetasone. Group III steroids include, but are not limited to, triamcinolone acetonide, mometasone furoate, fluticasone propionate, betamethasone dipropionate, and halometasone. Group IV steroids include, but are not limited to, fluocinolone acetonide, hydrocortisone valerate, hydrocortisone butyrate, flurandrenolide, triamcinolone acetonide, and mometasone furoate. Group V steroids include, but are not limited to, fluticasone propionate, desonide, fluocinolone acetonide, and hydrocortisone valerate. Group VI steroids include, but are not limited to, alclometasone dipropionate, triamcinolone acetonide, fluocinolone acetonide, and desonide. Group VII steroids include, but are not limited to, hydrocortisone (2.5%) and hydrocortisone (1%). The amount of hydrocortisone or steroid in the compositions may be from about 0.01% to about 5% (w/w), relative to the total amount of the composition or in some embodiments, from about 0.1% to about 1% (w/w), relative to the total amount of the composition, or any range or individual concentration encompassed in these example ranges.

In some embodiments, the compositions may further include an anti-inflammatory compound such as methotrexate, tofacitinib, 6-mercaptopurine, azathioprine sulphasalazine, mesalazine, olsalazine chloroquinine/hydroxychloroquinine, penicillamine, aurothiomalate (intramuscular and oral), azathioprine, cochicine, corticosteroids (oral, inhaled, and local injection), a beta-2 adrenoreceptor agonist (salbutamol, terbutaline, salmeteral), xanthine (theophylline, aminophylline), cromoglycate, nedocromil, ketotifen, ipratropium and oxitropium, cyclosporin, FK506, rapamycin, mycophenolate mofetil, leflunomide, an NSAID (e.g. ibuprofen), a corticosteroid (e. g. prednisolone), a phosphodiesterase inhibitor, an adensosine agonist, an antithrombotic agent, a complement inhibitor, an adrenergic agent, an agent that interferes with signalling by proinflammatory cytokines such as TNF or IL-1 (e.g., a NIK, IKK, p38 or MAP kinase inhibitor), an IL-1 converting enzyme inhibitor, a T-cell signalling inhibitor (e.g. a kinase inhibitor), a metalloproteinase inhibitor, sulfasalazine, a 6-mercaptopurine, an angiotensin converting enzyme inhibitor, a soluble cytokine receptor (e.g. soluble p55 or p75 TNF receptors and the derivatives p75TNFRigG (etanercept) and p55TNFRigG (Lenercept), siL-1RI, siL-1RII, siL-6R), an antiinflammatory cytokine (e.g. IL-4, IL-1 0, IL-11, IL-13 and TGF), celecoxib, folic acid, hydroxychloroquine sulfate, rofecoxib, etanercept, infliximab, adalimumab, certolizumab, tocilizumab, abatacept, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, cortisone, betamethasone, morphine sulfate, lidocaine hydrochloride, indomethacin, glucosamine sulf/chondroitin, amitriptyline HCl, sulfadiazine, oxycodone HCV acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL1S, BIRB-796, SC10-469, VX-702, AMG-548, VX-740, Roflumilast, IC-485, CDC-801, S1PI agonists (such as FTY720), a PKC family inhibitor (e.g. Ruboxistaurin or AEB-071) or Mesopram, budenoside; epidermal growth factor; a corticosteroid; cyclosporin, sulfasalazine; an aminosalicylate; 6-mercaptopurine; azathioprine; metronidazole; a lipoxygenase inhibitor; mesalamine; olsalazine; balsalazide; an antioxidant; a thromboxane inhibitor; an IL-1 receptor antagonist; an anti-IL-1 monoclonal antibody; an anti-IL-6 monoclonal antibody; a growth factor; an elastase inhibitor; a pyridinyl-imidazole compound; an antibody to or antagonist of other human cytokines or growth factors (e.g. TNF, LT, IL-1, IL-2, IL-6, IL-7, IL-8, IL-12, IL-15, IL-16, IL-23, EMAP-II, GM-CSF, FGF, and PDGF); a cell surface molecule (e.g. CD2, CD3, CD4, CD8, CD25, CD28, CD30, CD40, CD45, CD69, or CD90 or their ligands); methotrexate; cyclosporine; FK506; rapamycin; mycophenolate mofetil; leflunomide; an NSAID (e.g. ibuprofen); a corticosteroid (e.g. prednisolone); a phosphodiesterase inhibitor; an adenosine agonist; an antithrombotic agent; a complement inhibitor; an adrenergic agent; an agent that interferes with signalling by proinflammatory cytokines such as TNF 5 or IL-1 (e.g. a NIK, IKK, or MAP kinase inhibitor); an IL-1 converting enzyme inhibitor; a TNF converting enzyme inhibitor; a T-cell signalling inhibitor such as kinase inhibitors; a metalloproteinase inhibitor; sulfasalazine; azathioprine; a 6-mercaptopurine; an angiotensin converting enzyme inhibitor; a soluble cytokine receptor (e.g. soluble p55 or p75 TNF receptors, siL-1RI, siL-1RII, siL-6R), an antiinflammatory cytokine (e.g. IL-4, IL-1 0, IL-11, IL-13 or TGF), therapeutic agents that target an intrinsic checkpoint blockade, such as, for example, the gene encoding Cytokine-inducible SH₂-containing protein (CISH), antibody BGB-A317, Nivolumab, or Pembrolizumab, atezolizumab, avelumab, durvalumab, ipilimumab, and the like and combinations thereof. The amount of anti-inflammatory in the composition may be from about 0.01% to about 5% (w/w), relative to the total amount of the composition, or in some embodiments, from about 0.1% to about 1% (w/w), relative to the total amount of the composition, or any range or individual concentration encompassed by these example ranges.

In some embodiments, the compositions may further include an antibiotic. The antibiotic compound is not particularly limited, and antibacterial, antifungal, antiprotozoal, and other antimicrobial agents. In certain embodiments, the antibiotic may include, for example, ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, ticarcillin clavulanic acid, nafcillin, procaine penicillin, cefadroxil, cefazolin, cephalexin, cephalothin, cephapirin, cephradine, cefaclor, cefamandol, cefonicid, cefotetan, cefoxitin, cefprozil, ceftmetazole, cefuroxime, loracarbef cefdinir, ceftibuten, cefoperazone, cefixime, cefotaxime, cefpodoxime proxetil, ceftazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, perfloxacin, imipenem-cilastatin, meropenem, aztreonam, and the like and combinations thereof. Antifungal agent antibiotics include, for example, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, tolnaftate, undecylenic acid, crystal violet, balsam of Peru, and the like and combinations thereof. The amount of the antibiotic in the compositions may be from about 0.01% to about 5% (wt./w), relative to the total amount of the composition, or in some embodiments, from about 0.1% to about 1% (w/w), relative to the total amount of the composition, or any range or individual composition encompassed by these example ranges.

In some embodiments, the compositions may further include analgesic agent. The analgesic agent is not particularly limited and includes, for example, methyl salicylate, codeine, morphine, methadone, pethidine, buprenorphine, hydromorphine, levorphanol, oxycodone, fentanyl, a non-steroidal anti-inflammatory drug, and the like and combinations thereof. The amount of the analgesic agent in the compositions may be from about 0.01% to about 5% (w/w), relative to the total amount of the composition or in some embodiments, from about 0.1% to about 1% (w/w), relative to the total amount of the composition, or any range or individual concentration encompassed by these example ranges.

In some embodiments, the compositions may further include antiviral compound. The anti-viral compound is not particularly limited and includes, for example, acyclovir, famciclovir, penciclovir, valacyclovir, trifluridine, docosanol, amantadine, rimantadine, oseltamivir, and zanamivir. The amount of the anti-viral compound in the compositions may be from about 0.01% to about 5% (w/w), relative to the total amount of the composition or in some embodiments, from about 0.1% to about 1% (w/w), relative to the total amount of the composition, or any range or individual concentration encompassed by these example ranges.

Cannabinoids are generally insoluble in water. The compositions of the invention, therefore, may include one or more pharmaceutically acceptable solvent or co-solvents for the cannabinoid. The solvent may be present in an amount effective to have the cannabinoids substantially solubilized in the formulation. Therefore, the amount of solvent in the formulation will vary based on the concentration of the cannabinoid. The amount of solvent will also vary based on the partition coefficient of the particular cannabinoid molecule.

In certain embodiments, the solvents may be pharmaceutically acceptable organic solvents such as dehydrated alcohol, ethanol, propanol, isopropanol, propylene glycol, polyethylene glycol, and the like and combinations thereof. The amount of organic solvent in a particular composition may vary based on the intended formulation and the solubility of the cannabinoid. The amount of dehydrated alcohol in the formulations of the present invention can have a range of about 10% to about 90%; (w/w), about 15% to about 65% (w/w), about 15% to about 50% (w/w), or any range or independent concentration encompassed by these example ranges.

In some embodiments, the composition may include a co-solvent such as, for example, polyethylene glycol. In certain embodiments, the co-solvent may be a low molecular weight polyethylene glycol such as polyethylene glycol 400 (PEG 400). The co-solvent may have a concentration of about 1% to about 50% (w/w) of the composition, about 1% to about 15% (w/w), about 1% to about 10% (w/w) relative to the total amount of the composition, or any range or independent concentration encompassed by these example ranges. In various embodiments, the solvent and co-solvents may make up about 65% to about 75% (w/w) relative to the total amount of the composition

In some embodiments the compositions may further include a solubilizing agent. Solubilizing agents include, for example, Capryol 90, Cremophor RH40, Labrafil M 1944 CS, Labrafil M 2125 CS, Lauroglycol 90, PEG MW>4000, Plurol Oleique CC 497, poloxamer 124, poloxamer 188, Softigen 701, Softigen 767, Tagat TO, Tween 80, triacetin, triethylcitrate, tributylcitrate, acetyl triethylcitrate, acetyl tributyl citrate, ethyl oleate, ethyl caprylate, ethyl butyrate, triacetin, 2-pyrrolidone, 2-piperidone, N-methylpyrrolidone, N-ethylpyrrolidone, N-hydroxyethyl pyrrolidone, N-octylpyrrolidone, N-laurylpyrrolidone, dimethylacetamide, Miglyol, lanolin, petrolatum, mineral oil, and the like and combinations thereof. The solubilizing agent may have a concentration of about 5% to about 85% (w/w) of the total inactive ingredients.

Other components such as preservatives, antioxidants, surfactants, absorption enhancers, viscosity modifiers, bulking agents, diluents, coloring agents, flavoring agents, pH modifiers, sweeteners or taste-masking agents may also be incorporated into any of the compositions of the invention. The amount of each of these components which may be used will be optimized for each formulation, in order to obtain a stable product (dosage form) having the desired shelf-life. Generally suitable formulations may include from about 0.001% to about 20% (w/w) of a pharmaceutically acceptable preservative, antioxidant, surfactant, absorption enhancer, viscosity modifier, bulking agent, diluent, coloring agent, flavoring agent, pH modifier, sweetener or taste-masking agent.

In some embodiments, the compositions may include an effective (stabilizing) amount of one or more pharmaceutically acceptable antioxidants. Any of the known antioxidants may be used, including but not limited to antioxidants such as butylated hydroxyanisole (BHA), butylated hydroxytoluene (BHT), propyl gallate, lecithin, Vitamin E tocopherol, sesamin, sesamol, sesamolin, alpha tocopherol, ascorbic acid, ascorbyl palmitate, fumaric acid, malic acid, sodium ascorbate and sodium metabisulphite, as well as chelating agents such as disodium EDTA, may also be used to stabilize the cannabinoid formulations of the present invention. The compositions may also include antioxidant synergists to prevent oxidative degradation. Such antioxidant synergists include, for example, disodium edetate and the like and combinations thereof. Such compositions may include about 0.001% to about 20% (w/w) antioxidant, about 0.001 to about 1% (w/w), about 0.01% to about 0.1% (w/w) relative to the total amount of the composition, or any range or individual concentration encompassed by these example ranges.

In some embodiments, the compositions may further include one or more organic or inorganic bases. Examples of suitable organic bases include, but are not limited to, any pharmaceutically acceptable primary, secondary and tertiary organic amines, such as methanolamine, ethanolamine, meglumine, other alkylamines (e.g. di-alkyl amines and tri-alkyl amines), and the like and combinations thereof. Inorganic bases include, for example, sodium hydroxide (NaOH), magnesium hydroxide (MgOH) and the like and combinations thereof. In such embodiments, the organic or inorganic bases may have a concentration of about 0.001% to about 20% (w/w), about 0.01% to about 15% (w/w), about 0.01% to about 5% (w/w), about 0.05% to about 2% (w/w), or any range or individual value encompassed by these example ranges. In some embodiments, the composition may include both an organic base and an inorganic base.

Preservatives typically have bactericidal and fungicidal properties. For example, parabens are widely used preservatives in the pharmaceutical industry. In some embodiments, the compositions may include a combination of parabens which may allow for the use of lower concentrations of preservative with increasing preservative activity. Examples of parabens include methyl paraben, propyl paraben, and the like and combinations thereof. In various embodiments, the compositions of the invention may include about 0.001% to about 5.0% (w/w), about 0.001% to about 1.0% (w/w), about 0.01% to about 0.05% (w/w) paraben relative to the total amount of the composition, or any range or individual concentration encompassed by these example ranges. In some embodiments, the composition may include a combination of parabens, such as about 0.001% to about 0.1% (w/w) methyl paraben and about 0.001% to about 0.1% (w/w) propyl paraben, or any range or individual concentration of methyl or propyl paraben encompassed by these ranges.

In some embodiments, the compositions may further include a physiologically acceptable buffer the pH to maintain the composition at a physiologically compatible pH range, enhance the solubility and stability of the cannabinoid, and the like. Suitable buffers include, but are not limited to acetate, bicarbonate, citrate, phosphate, pharmaceutically acceptable salts thereof and combinations or mixtures thereof. The compositions of the invention may include about 0.1% to about 20% (w/w), about 0.5% to about 10% (w/w), or any range or individual concentration encompassed by these example ranges. The concentration of buffer may be such that the composition has a pH of about 5 to about 10, about 6 to about 8, or about 7.

In certain embodiments, the composition may be isotonic. Isotonic formulations may be provided by the addition of a tonicity agent. Suitable tonicity agents include, but are not limited to any pharmaceutically acceptable sugar such as dextrose, salt, i.e., sodium chloride, and the like and combinations or mixtures thereof. The compositions may have a concentration of tonicity agent of about 100 mOsm/kg to about 500 mOsm/kg, about 200 mOsm/kg to about 400 mOsm/kg, about 250 mOsm/kg to about 300 mOsm/kg, or any range or individual concentration encompassed by these example ranges.

In some embodiments, the composition may include a sweetener such as, for example, sucrose, fructose, sucralose, sorbitol, xylitol, saccharin, saccharin sodium, and the like and combinations thereof. The concentration of sweetener may be about 0.01% to about 25% (w/w), about 0.01% to about 10% (w/w), about 0.01% to about 5% (w/w), or any range or individual concentration encompassed by these example ranges.

In further embodiments, the compositions may include a viscosity modifier such as, for example, cellulose or cellulose derivatives such as ethylcellulose, hydroxyethylcellulose, hydroxypropylcellulose, caboxymethylcellulose, sodium hydroxypropylmethylcellulose, methylcellulose, methylethylcellulose, sodium carboxymethylcellulose, Aerosil (silicon dioxide), cetostearyl alcohol, cetyl alcohol, stearyl alcohol, Gelucires 33/01, 39/01 and 43/01, glyceryl behenate (Compritol 888 A TO), glyceryl palmitostearate (Precirol AT05), Softisans 100, 142, 378 and 649, stearyl alcohol carbomer, xanthan gum, maltodextrin, acacia, tragacanth, povidone and polyvinyl alcohol. The concentration of viscosity modifier may be about 0.01% to about 25% (w/w), about 0.01% to about 10% (w/w), about 0.01% to about 5% (w/w), or any range or individual concentration encompassed by these example ranges.

In some embodiments, the compositions may further include an absorption enhancer such as, for example, Gelucire 44/14, Gelucire 50/13, Tagat TO, Tween 80, isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, and the like and combinations thereof. In certain preferred embodiments, the absorption enhancer may be triacetin. The absorption enhancer in the compositions may have a concentration of may be about 0.01% to about 25% (w/w), about 0.01% to about 10% (w/w), about 0.01% to about 5% (w/w) relative to the total amount of the composition, or any range or individual concentration encompassed by these example ranges.

In some embodiments, the compositions may include a bulking agent such as, for example, microcrystalline cellulose, mannitol, xylitol, starches, and the like and combinations thereof. In such embodiments, the bulking agent may have a concentration of about 0.01% to about 25% (w/w), about 0.01% to about 10% (w/w), about 0.01% to about 5% (w/w), or any range or individual concentration encompassed by these example ranges.

In some embodiments, the compositions of the invention may contain a gelling or suspension agent such as, for example carbomers such as Carbopol, modified cellulose derivatives, naturally-occurring, synthetic or semi-synthetic gums such as xanthan gum, acacia and tragacanth, modified starches, co-polymers such as those formed between maleic anhydride and methyl vinyl ether, colloidal silica and methacrylate derivatives sold under the trade name Eudragit™, and the like and combinations thereof. In various embodiments, the gelling or suspension agent may have a concentration of about 0.01% to about 25% (w/w), about 0.01% to about 10% (w/w), about 0.01% to about 5% (w/w), or any range or individual concentration encompassed by these example ranges.

In further embodiments, additional excipients may be incorporated into the compositions of the invention as needed, including, for example, surfactants (e.g. Capryol 90; Cremophor RH40; Gelucire 44/14; Gelucire 50/13; Imwitor 91; Imwitor 308; Imwitor 380; Imwitor 742; Imwitor 780K; Imwitor 928; Imwitor 988; Labrafil M 1944 CS; Labrafil M 2125 CS; Lauroglycol 90; Tagat TO; Tween 80; and mixtures thereof); and emulsifiers (e.g., Gelucire 44/14; Gelucire 50/13; Imwitor 91; Imwitor 308; Imwitor 380; Imwitor 742; Imwitor 780K; Imwitor 928; Imwitor 988; poloxamer 124; poloxamer 188; Tagat TO; Tween 80; lecithin; lysolecithin; phosphatidylcholine; phosphatidylethanolamine; phosphatidylglycerol; phosphatidic acid; phosphatidylserine; lysophosphatidylcholine; lysophosphatidylethanolamine; lysophosphatidylglycerol; lysophosphatidic acid; lysophosphatidyl serine; PEG-phosphatidylethanolamine; PVP-phosphatidylethanolamine; sodium lauryl sulfate and mixtures thereof).

Other additives conventionally used in pharmaceutical compositions can be included, and these additives are well known in the art. Such additives include pharmaceutically acceptable detackifiers, anti-foaming agents, chelating agents, viscomodulators, tonicifiers, flavorants, colorants odorants, opacifiers, suspending agents, binders, fillers, plasticizers, lubricants, and mixtures thereof. The amounts of such additives can be readily determined by one skilled in the art, according to the particular properties desired, keeping in mind the possibility that any such additives should preferably not negatively impact the stability of the final formulation.

Suitable coloring agents include red, black and yellow iron oxides and FD&C dyes such as FD&C Blue No. 2, FD&C Red No. 40, and the like. Suitable flavoring agents include mint, raspberry, licorice, orange, lemon, grapefruit, caramel, vanilla, cherry grape flavors, combinations thereof, and the like. Suitable sweeteners include sucralose, xylitol, saccharin, and the like. Suitable pH modifiers include citric acid, tartaric acid, phosphoric acid, hydrochloric acid, maleic acid, sodium hydroxide, and the like. Suitable sweeteners include aspartame, acesulfame K, thaumatic, and the like. Suitable taste-masking agents include sodium bicarbonate, ion-exchange resins, cyclodextrin inclusion compounds, adsorbates, and the like.

It is recognized that pharmaceutical excipients may perform more than one function, and are therefore characterized as having different uses depending on the particular application. While the use of an excipient in the context of a particular formulation may determine the function of the excipient, the inclusion of any particular excipient into any one or more category as set forth above is not meant to limit the function of that excipient.

Although the ingredients of the formulations of the present invention are characterized herein as percentage based on weight, one skilled in the art will appreciate that scaled-up versions of the formulations specifically described herein may be characterized instead on a volume percentage basis. Where the density of a particular component is 1 g/ml the amount of the component based on volume and weight will be the same. Where the density deviates from 1 g/ml, the amounts based on weight or volume will differ accordingly.

Another embodiment of the present invention is a method of making the composition in the form of a tincture. Tinctures are herbal extracts that provide a method for oral administration of an herbal component of components to a subject in need of treatment. Tinctures are prepared by mixing an herb or herbs or components or combinations thereof with a suitable solvent wherein a component or components of an herb or combinations thereof are extracted into a solvent in which the component or components of the herbs are reasonably soluble. Suitable tincture solvents in the present invention include pharmacologically acceptable solvent such as organic solvents, water based solvents, alcohols, and other orally administrable solvents such as, but not limited to, water, purified water, preserved water, vegetable glycerin, propylene carbonate, 3-methoxy-3-methyl-1-butanol (MMB), polyethylene glycerol, rice bran oil, and combinations thereof.

In some embodiments, the composition can be in the form of a tonic. Tonics are extracts that provide a method for oral administration of an herbal component or components to a subject in need of treatment. Tonics are prepared by mixing an herb or herbs or components or combinations thereof with a suitable solvent wherein a component or components of an herb or herbs or combinations thereof are extracted into a solvent by aid of heating, often heat necessary such that the solvent reaches its boiling temperature, in which the component or components of the herb are reasonably soluble. Suitable tonic solvents in the present invention include pharmacologically acceptable solvents such as organic solvents, water based solvents, alcohols, and other orally administrable solvents such as, but not limited to, water, purified water, preserved water, vegetable glycerin, propylene carbonate, 3-methoxy-3methyl-1-butanol (MMB), polyethylene glycol, rice bran oil, and combinations thereof.

In some embodiments, the composition can be in the form of a tablet. Tablets are pharmaceutical oral doses of a medicament or medicaments that are formed by molding or compression. Such embodiments are comprised of the medicament or medicaments and may be further comprised of suitable excipients such as, but not limited to, diluents, binders, granulating agents, gildants, lubricants, disintegrants, sweeteners, and pigments. Tablets in the present invention may also be coated with a pigment to increase the visual appearance of the tablet, to increase the identifiability of the tablet, to increase the ease with which the tablet is orally administered, to make the tablet more easily swallowed, to control the release of the medicament or medicaments, or to make the tablet more resistant to environmental degradation factors, or a combination or combinations thereof.

In another embodiment, the composition can be in the form of a capsule. Capsules generally fall within the class of either hard-shelled capsules or soft-shelled capsules, but need not be restricted to either class. Hard shelled capsules generally, but need not necessarily, contain dry, powdered, or granular components while soft-shelled capsules primarily, but need not necessarily, contain oils or medicaments or combinations thereof.

Another embodiment of the present invention is a method of treating various disease states, which comprises administering a therapeutically effective amount of the compositions described invention to a patient in need of treatment. In various embodiments, the patient may suffer from chronic medical condition or symptoms relating to chronic medical conditions including for example, inflammatory diseases, autoimmune diseases, heart disease, obesity, and the like. Non-limiting examples of the diseases that can be treated using the methods of the invention include, for example, arteriosclerotic heart disease, arthritis, rheumatoid arthritis, asthma, gout, attention deficit hyperactivity disorder (ADD/ADHD), autism, Ssperger's syndrome, chronic pain, substance dependence, diabetic neuropathy, glaucoma, migraine headaches, tension headaches, hypertension, inflammatory autoimmune-mediated arthritis, inflammatory bowel disease (IBD), insomnia, depression, obesity, obsessive compulsive disorder, opiate dependence, osteoarthritis, post-traumatic stress disorder (PTSD), restless legs syndrome (RLS), rheumatoid arthritis, seizures, dementia, nausea, sleep disorders, tourette's syndrome, fibromyalgia, and related conditions, epilepsy, digestive diseases, gliomas, and the like and combinations thereof.

In some embodiments, the method may include tapering doses of cannabidiol. Thus, embodiments may include a treatment regimen in which compositions containing different concentrations of cannabidiol are administered over the course of treatment. For example, some embodiments include administered one or more doses of a first composition containing greater than 10% (w/w) cannabidiol to a subject in need of treatment then subsequently administering one or more doses of a second composition containing less cannabidiol than the first topical composition to the subject in need of treatment. For example, if the first composition contains about 20% (w/w) cannabidiol, the second composition may include 19% (w/w) or less cannabidiol. In further embodiments, the method may include administering one or more doses of a third composition containing less cannabidiol than the second composition, a fourth composition containing less cannabidiol than the third composition, and so on. In particular embodiments, the method may include administering a composition containing a maintenance dose of cannabidiol that is equal to or less than the final dosage of a dosage regimen containing two or more compositions of decreasing dosage. The maintenance dose may provide sufficient cannabidiol to reduce or eliminate potential recurrence of the dermatological disease.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

Solid Capsule with Bioenhancer

Capsules containing solid CBD, 10 mg of the natural bioenhancer piperine, and inactive excipients were prepared by mixing and filling into gel caps. These were distributed among a group of 12 habitual CBD users along with gelcaps not containing a bioenhancer.

Six habitual CBD users were administered two bioenhancer:CBD gel caps in the morning and evening. Six habitual CBD users were administered two CBD only gel caps in the morning and evening. After one week of use the group was surveyed on the effectiveness of the bioenhancer. All six members of the CBD:bioenhancer group reported much greater effectiveness while all six members of the CBD only pills reported no increase in effect.

### Example 2

Liquid Formulations with Bioenhancers—

Tinctures containing 500 mg of CBD, 20 mg/mL of the natural bioenhancer quercetin, and inactive excipients/carrier oils were prepared by mixing and filling into 30 mL bottles.

Six habitual CBD users were administered two bioenhancer:CBD gel caps in the morning and evening. Six habitual CBD users were administered two CBD only gel caps in the morning and evening. After one week of use the group was surveyed on the effectiveness of the bioenhancer. All six members of the CBD:bioenhancer group reported much greater effectiveness while all six members of the CBD only pills reported no increase in effect.

1. A composition comprising a cannabinoid, a bioenhancer having a concentration of about 0.05% (w/w) to about 30% (w/w), relative to the total weight of the composition, and a pharmaceutically acceptable carrier, excipient, diluent, reagent, or combinations thereof.

2. The composition of claim 1, wherein the cannabinoid has a concentration of about 0.5% (w/w) to about 20% (w/w), relative to the total amount of the composition.

3. The composition of claim 1, wherein the cannabinoid is selected from the group consisting of cannabidiol, cannabidiol isomer, cannabidiol analog, and combinations thereof.

4. The composition of claim 4, wherein the bioenhancer comprises a P-glycoprotein inhibitor selected from the group consisting of piperine, quercetin, genistein, naringin, sinomenine, glycyrrhizin, nitrile glycosides cuminum cyminum, zingiver officinale, lysergol, allium sativum, aloe vera, and combinations thereof.

5. The composition of claim 4, wherein the bioenhancer is selected from the group consisting of liposomes, microspheres, nanoparticles, transferosomes, ethosomes, nanoemulsions, microemulsions, lipid based systems, polymeric micelle formulations, ketoprofen-loaded solid lipid nanoparticles, beeswax, carnauba wax, natural waxes, solid lipids, and combinations thereof.

6. The composition of claim 4, wherein the bioenhancer is selected from the group consisting of Ginkgo biloba lipid-based systems, silybin lipid-based systems, ginseng lipid-based systems, hawthorn lipid-based systems, quercetin lipid-based systems, curcumin lipid-based systems, and combinations thereof.

7. The composition of claim 4, wherein the bioenhancer is selected from the group consisting of capsaicin transferosomes, colchicine tranferosomes, vincristine tranferosomes, and combinations thereof.

8. The composition of claim 1, further comprising a steroid, anti-inflammatory, immune checkpoint blockade inhibitor, antibiotic, antiviral compound, or combinations thereof.

9. The composition of claim 11, wherein the amount of steroid, anti-inflammatory, immune checkpoint blockade inhibitor, antibiotic, or antiviral compound, or combinations thereof may be from about 0.01% to about 50% (wt/wt), relative to the total amount of the composition.

10. The composition of claim 1, wherein the composition is in the form of a gel, tonic, tincture, pills, tablet, capsule, or combinations thereof.

11. A method for treating a disease, comprising administering to a patient in need of treatment composition comprising a cannabinoid having a concentration, a bioenhancer having a concentration of about 0.05% wt to about 20% wt, relative to the total weight of the composition, and a pharmaceutically acceptable carrier, excipient, diluent, reagent, or combinations thereof.

12. The method of claim 12, wherein the disease is selected from the group consisting of inflammatory diseases, autoimmune diseases, heart disease, obesity, and combinations thereof.

13. The method of claim 12, wherein the disease is selected from the group consisting of arteriosclerotic heart disease, arthritis, rheumatoid arthritis, asthma, gout, attention deficit hyperactivity disorder (ADD/ADHD), autism, Ssperger's syndrome, chronic pain, substance dependence, diabetic neuropathy, glaucoma, migraine headaches, tension headaches, hypertension, inflammatory autoimmune-mediated arthritis, inflammatory bowel disease (IBD), insomnia, depression, obesity, obsessive compulsive disorder, opiate dependence, osteoarthritis, post-traumatic stress disorder (PTSD), restless legs syndrome (RLS), rheumatoid arthritis, seizures, dementia, nausea, sleep disorders, tourette's syndrome, fibromyalgia, and related conditions, epilepsy, digestive diseases, gliomas, and the like and combinations thereof.

14. The method of claim 12, wherein the concentration of the cannabinoid has a concentration in the range of about 1% to about 30% (w/w), relative to the total amount of the composition.

15. The method of claim 12, wherein the cannabinoid is selected from the group consisting of cannabidiol, cannabidiol isomer, cannabidiol analog, or combinations thereof.

16. The method of claim 12, wherein the cannabinoid has a concentration in the range of about 5% to about 20% (w/w), relative to the total amount of the composition.

17. The method of claim 12, wherein the bioenhancer comprises a P-glycoprotein inhibitor selected from the group consisting of piperine, quercetin, genistein, naringin, sinomenine, glycyrrhizin, nitrile glycosides cuminum cyminum, zingiver officinale, lysergol, allium sativum, aloe vera, and combinations thereof.

18. The method of claim 12, wherein the composition further comprises a steroid, anti-inflammatory, immune checkpoint blockade inhibitor, antibiotic, antiviral agent, or combinations thereof.
